(19) United States
(12) Patent Application Publication
(10) Pub. No.: US 2010/0233137 A1
(43) Pub. Date: Sep. 16, 2010

(54) Compositions and Methods for Modification of Target Cells and to Their Uses Thereof

(75) Inventor: Jeffrey John Thramann, Niwot, CO (US)

Correspondence Address: SWANSON & BRATSCHUN, L.L.C., 8210 SOUTHPARK TERRACE, LITTLETON, CO, 80120, US

(73) Assignee: LANX, INC., Broomfield, CO (US)

(21) Appl. No.: 12/528,481

(22) PCT Filed: Feb. 27, 2008
(86) PCT No.: PCT/US08/55061

§ 371 Date: Dec. 14, 2009

Related U.S. Application Data
(60) Provisional application No. 60/891,850, filed on Feb. 27, 2007.

(51) Int. Cl. A61K 35/12

(52) U.S. Cl. 424/93.7

(57) ABSTRACT

Methods and compositions are described for preparing modified cells useful in various therapeutic applications.

### TECHNICAL FIELD

The invention generally relates to methods and compositions for generating target cells useful in various therapeutic applications. More particularly, the invention relates to modifying target factors in stem and other like cells in order to enhance the capacity of the cells to differentiate into target tissues and cell types.

### BACKGROUND OF THE INVENTION

The use of stem cell based therapies in regenerative medicine provides great potential to the health care industry. Stem cell research has focused on use of these cells in replacement and repair of organs and tissues in patients in need thereof. Stem cell therapies have been proposed for treatment of Parkinson's disease, Alzheimer's disease, stroke, heart disease, osteoarthritis, osteoporosis, rheumatoid arthritis, diabetes, disc degeneration, spine fusion, and other like diseases.

Research into stem cell based regenerative medicine has focused on both the properties that determine how stem cells remain unspecialized and self renewing, and on identifying the conditions necessary to cause stem cells to become specialized or differentiated, e.g., what conditions must prevail in order to turn a stem cell into an osteoblast, a chondrocyte, a neuron, etc. The control of these two processes, cell renewal and cell differentiation, are necessary to improving regenerative therapies.

Mesenchymal stem cells (MSCs) are pluripotent cells found in at least bone marrow, adipose tissue, blood, dermis, hair follicles, and periosteum. MSCs are capable of differentiating into various mesenchymal and/or connective tissues including osseous, muscular, adipose, cartilaginous, and fibrous. Recent data has also shown the ability of MSC's to differentiate into other tissues typically not associated with the mesoderm, for example, MSC's have now been differentiated into neural tissue. In recent years studies have been conducted identifying various factors useful in facilitating the targeted differentiation of MSCs into a particular organ or tissue, for example, bone morphogenetic protein 2 (BMP-2) expression in MSCs facilitates in vivo osteochondral differentiation. see for example Noël et al, Stem Cells 2004; 22:74-85. Various labs have attempted to modify MSCs to be used in restoration of target organs and tissues, this research has generally focused on identification of factors or conditions required to induce differentiation of stem cells into target tissue, as well as establishment of stable cell lines for use in research and potential health issues.

Against this backdrop the present disclosure is presented.

### SUMMARY OF THE INVENTION

The present invention provides compositions and methods for preparing and utilizing modified cells, e.g., modified stem cells, modified mesenchymal stem cells, modified chondrocytes, modified osteoblasts, etc, for various therapeutic applications.

In one aspect, cells are modified using non-viral methods to incorporate useful factors, for example, transcription factors, into stem cells and then implanting these cells to achieve a desired therapeutic result. Non-viral modifications include use of polymeric vesicles to encapsulate and deliver various factor polypeptides to the target cell. These modified cells (for example modified stem cells) have an increased capacity to differentiate or transition or function as a desired cell type useful in the therapeutic application. In one example, the transcription factor is a SMAD protein (Sma mad Mad related protein) and the target cell is a stem cell, for example a mesenchymal stem cell. Other non-viral delivery methods are contemplated, for example through the use of carrier liposomes or an electrical current to open the cell membrane and allow access of the factors to the target cell. In each instance the modified cells, i.e., cells that have incorporated the factor polypeptide, have enhanced capacity in the environment of the therapeutic application as compared to implanted unmodified cells or to native cells to the environment.

In another aspect, the above described results are obtained by using viral based genetic engineering techniques to incorporate useful factors, for example, transcription factors, into target cells and then implanting these cells to achieve a desired therapeutic result. Viral based techniques include preparing stably or transiently transfected cells that overexpress the useful factor. In one embodiment the useful factor is a SMAD protein.

In another aspect, the modified cells are treated with an appropriate signaling factor to stimulate the incorporated factor to further enhance the desired modification of the target cell and ultimately the desired therapeutic application. In one embodiment non-virally modified mesenchymal stem cells having enhanced levels of SMAD proteins are treated with BMP-2 prior to implantation into a degenerated disc. Treatment with BMP-2 facilitates functional activation of the modified MSCs.

Embodiments of the invention include incorporation of one or more, two or more, or a plurality of incorporated factors to modify the target cells for a therapeutic application. Where cells are modified to include more than one factor, embodiments are generally designed to include factors that benefit each other in obtaining the desired result, i.e. participate together to obtain the same result in a common signal transduction pathway. For example, modifying cells to include a SMAD protein and a kinase that phosphorylates the SMAD protein thereby signaling the SMAD to translocate to the nucleus and activate transcription useful in the differentiation of the cell into a more useful therapeutic cell. In addition, delivery methods can be utilized wherein known complexes of proteins are preformed prior to incorporation into the target cells, for example, complexes including Smad1, Smad4 and a transcription factor, e.g., Cbfa-1.

Embodiments of the invention also include preparing modified cells through a combination of the two general delivery methods, i.e., non-viral and viral. In these embodiments a target cell is genetically engineered to express a useful factor and then further prepared using a non-viral method to further include another useful factor. One example includes genetically engineering a mesenchymal stem cell to over express Smad2, which cells are then incubated with Smad4 loaded polymeric vesicles. The Smad2, Smad4 mesenchymal stem cells are then ready for therapeutic application (either in the presence or not of BMP-2).

These and various features and advantages of the invention will be apparent from a reading of the following detailed description and a review of the appended claims.

### DETAILED DESCRIPTION OF THE INVENTION

### Definitions

The following definitions are provided to facilitate understanding of certain terms used frequently herein and are not meant to limit the scope of the present disclosure.

“Amino acid” refers to any of the twenty naturally occurring amino acids as well any modified amino acid sequences. Modifications may include natural processes such as posttranslational processing, or may include chemical modifications which are known in the art. Modifications include but are not limited to: phosphorylation, ubiquitination, acetylation, amidation, glycosylation, covalent attachment of flavin, ADP-ribosylation, cross-linking, iodination, methylation and the like.

“Fusion protein” refers to a first peptide having attached a second, heterologous peptide. Preferably, the heterologous peptide is fused via recombinant DNA technologies, such that the first and second peptides are expressed in frame. The heterologous peptide can confer a desired characteristic to the fusion protein, for example, a detection signal, enhanced stability or stabilization of the protein in the cell, facilitated oligomerization of the peptide, or facilitated purification of the fusion proteins. Examples of heterologous peptides useful in the fusion proteins of the invention include transduction domains, immunoglobulin molecules, peptide tags, leucine zipper domains, cytokines, signal peptides, and the like.

“Genetically engineered” refers to any recombinant DNA or RNA method used to create a host cell that expresses a target protein at elevated levels, at lowered levels, or in an altered non-native form. In other words, the host cell has been transfected, transformed, or transduced with a recombinant polynucleotide molecule, and thereby been altered so as to cause the cell to alter expression of the desired protein. Methods and vectors for genetically engineering host cells are well known in the art, for example, various techniques are illustrated in Current Protocols in Molecular Biology, Ausubel et al., eds. (Wiley & Sons, New York, 1988, and quarterly updates). In addition, genetically engineering techniques include but are not limited to expression vectors, targeted homologous recombination and gene activation (see, for example, U.S. Pat. No. 5,272,071) and trans activation by engineered transcription factors (see for example, Segal et al., 1999, Proc Natl Acad Sci USA 96(6):2758-63).

“Host cell(s)” refers to cells capable of modification by methods and compositions of the present invention. In some embodiments the host cell(s) is a stem cell, for example a mesenchymal stem cell. Host cells can be established in ex vivo culture.

“Identity” refers to a comparison between pairs of nucleic acid or amino acid molecules. Methods for determining sequence identity are known. See for example, computer programs commonly employed for this purpose, such as the Gap program (Wisconsin Sequence Analysis Package, Version 8 for Unix, Genetics Computer Group, University Park, Madison Wis.), that uses the alogorithm of Smith and Waterman, 1981, Adv. Appl. Math., 2:482-489.

“Nucleic acid sequence” refers to the order or sequence of deoxyribonucleotides along a strand of deoxyribonucleic acid. The order of these deoxyribonucleotides determines the order of amino acids along a polypeptide chain. The deoxyribonucleotide sequence thus codes for the amino acid sequence.

“Protein,” “peptide,” and “polypeptide” are used interchangeably to denote an amino acid polymer or a set of two or more interacting or bound amino acid polymers.

“Selectable marker” refers to a marker that identifies a cell as having undergone a recombinant DNA or RNA event. Selectable markers include, for example, genes that encode antimetabolite resistance such as the DHFR protein that confers resistance to methotrexate (Wigler et al., 1980 PNAS USA 77:3567), the GPT protein that confers resistance to mycophenolic acid (Mulligan and Berg, 1981 PNAS USA 78:2072), the neomycin resistance marker that confers resistance to the aminoglycoside G-418 (Calberre-Garapin et al., 1981, J Mol. Biol., 150:1), the Hygro protein that confers resistance to hygromycin (Santerre et al., 1984, Gene 30:147). And the Zeocin™ resistance marker (Invitrogen). In addition, the hypoxanthine-guanine phosphoribosyltransferase and adenine phosphoribosyltransferase genes can be employed in hgprt⁻ and aprt⁻ cells, respectively.

“SMADs” refer to evolutionary conserved proteins that directly, or in conjunction with a SMAD colocator protein, i.e., Smad4, translocate to the nucleus upon phosphorylation and activate target genes involved with the differentiation or modification of cells into cells more useful in various therapeutic applications. If not enumerated, the term SMAD refers to Smads 1, 4, 5 and 8. References for SMADs include: Wang et al (2005) Biochem J 386: 29-34; Massague et al (2005) Genes Dev 19:2783-2810; and Conaway R C, et al (2005) Trends Biochem Sci 30: 250-255, each of which is incorporated by reference herein.

“Stem cells” refer to primal cells common to multi-cellular organisms, stem cells retain the ability to differentiate into a wide variety of specialized cell types. Stem cells can be categorized as embryonic or adult (including stem cells obtained from umbilical cord blood and placenta). In one embodiment in accordance with the present invention the stem cells are mesenchymal stem cells (see for example U.S. Pat. No. 5,486,359, U.S. Pat. No. 6,261,549 and U.S. Pat. No. 5,811,094, which are each incorporated by reference herein for all purposes).

“Vector, “extrachromosomal vector” or “expression vector” refers to a first polynucleotide molecule, usually double-stranded, which may have inserted into it a second polynucleotide molecule, for example a foreign or heterologous polynucleotide. The heterologous polynucleotide molecule may or may not be naturally found in the host cell, and may be, for example, one or more additional copy of the heterologous polynucleotides naturally present in the host genome. The vector is adapted for transporting the foreign polynucleotide molecule into a suitable host cell. Once in the host cell, the vector may be capable of integrating into the host cell chromosomes. The vector may optionally contain additional elements for selecting cells containing the integrated polynucleotide molecule as well as elements to promote transcription of mRNA from transfected DNA. Examples of vectors useful in the methods of the present invention include, but are not limited to, plasmids, bacteriophages, cosmids, retroviruses, and artificial chromosomes.

The present invention provides methods and compositions for modifying signal transduction pathways in target cells in order to prepare modified cells useful in obtaining targeted therapeutic results. Typical target cells for use in accordance with the invention are stem cells (embryonic or adult), for example mesenchymal stem cells. Other target cells can be used where the therapeutic result is related to a more differentiated cell, for example, chondrocytes useful in production of cartilage or osteoblasts useful in production of bone. In a general sense, these various cell types will be referred to generically as “target cells.”

In one aspect, target cells are modified to include greater levels of useful factors, for example transcription factors, useful in modifying cells for a targeted therapeutic result. In one embodiment, the transcription factor(s) signal target cells to differentiate into modified cells having enhanced capacity for obtaining a therapeutic result(s) as compared to unmodified cells or native cells used under like conditions.

Several differentiation inducing transcription factors are disclosed for use in a target cell. These factors include, but are not limited to, SMAD proteins (typically Smad1, Smad4, Smad5 and Smad8), and Cbfa1 protein. Other classes of proteins are also envisioned to be within the scope of the present invention, including kinases, phosphatases, nuclear transport proteins, heat shock proteins, and the like. In addition, known factors useful in antagonizing signal transduction blocking factors (typically signaling transduction blocking factors involved in blocking differentiation of a cell to a more specialized cell) are within the scope of the present invention. For example, modifying target cells with antagonists to the YY1 transcription factor to unblock signal transduction pathways thereby leading to beneficial uses of the cells in targeted therapeutic applications (see Kurisaka A., et al., Molecular and Cellular Biology, 2003, vol 23, p 4494-4510).

In one embodiment, the target cells are mesenchymal stem cells. The mesenchymal stem cells (MSCs) can be autologous or allogenic (to the patient in need of the therapy) and can be obtained from blood, adipose, bone marrow, hair follicles, or other like tissue. MSCs are modified (see below) to include functional SMAD protein(s) which are then implanted into a joint having a cartilage defect. These modified mesenchymal stem cells have increased ability/capability to differentiate and function as chondrocytes based on the pre-additive effects of the incorporated SMAD protein(s). The modified cells are particularly useful in situations where the target site where the modified cells will be implanted has diminished function, for example a site in a patient having osteoarthritis or disc degeneration.

In some embodiments target cells are modified through incorporation of one or more different factor polypeptide(s). Typically the incorporated one or more factor polypeptide(s) is in a functional form, e.g., a form that is capable of initiating signal transduction within the target cells. For example, various transcription factors require phosphorylation, dephosphorylation, complex formation, folding, etc to translocate to the nucleus and activate or inactivate gene transcription necessary to induce target cell modification useful in the various therapeutic applications. In addition, where the one or more factor polypeptide(s) is an antagonist of a signal blocking factor, it also is typically in a form capable of performing its function.

In some embodiments target cells are modified through incorporation of two or more different transcription factor polypeptides, each directed at facilitating a signal transduction pathway necessary to modify a target cell for a therapeutic use. One example of this is to incorporate SMAD polypeptides in combination with other known SMAD polypeptide co-factors, e.g., P300, PCAF, GCN5, MSG1, etc. These combinations are provided to limit situations where an increase in one transcription factor is ineffective because the transcription factors co-factor is present in limiting levels. Other proteins involved in signal transduction pathway signaling can be incorporated, for example, kinases, phosphatases, nuclear transport proteins, DNA binding proteins, heat shock proteins, and the like.

Aspects of the invention include utilizing non-viral mechanisms to modify target cells with the factor(s) required of the invention. For example, embodiments of the invention include using polymeric vesicles, liposomes or other like carrier materials to encapsulate agents useful in modifying signal transduction pathways, for example for encapsulating transcription factors, to facilitate transporting them inside the target cells. The polymeric vesicles can be polymeric vesicles composed of polyarginine and polyleucine segments. These amino acid segments direct structure for vesicle formation and provide functionality for efficient intracellular delivery of the vesicles. Formation of the vesicles and loading with factor(s) is as described in Nat. Mater. 2007 Jan; 6(1):52-7, which is hereby incorporated by reference for all matters. Other carrier methods are also envisioned including liposome encapsulation. Formation of liposome encapsulated factors is as described in or using similar techniques as described in Szoka et al., Ann. Rev. Biophys. Bioeng. 9:467 (1980); U.S. Pat. Nos. 7,150,882 and 7,135,189, which are each incorporated by reference for all matters. Another method for incorporating proteins into target cells is through the use of protein transduction domains which are proteins that typically contain guanidine rich segments conjugated with drugs, oligonucleotides, proteins, nanoparticles, liposomes, etc for the purpose of delivering these items into target cells as described in Bogoyevitch et al., DNA and Cell Biology; Taking the Cell by Stealth or Storm? Protein Transduction Domains (PTDs) as Versatile Vectors for Delivery. Dec. 2002, Vol. 21, No. 12:879-894.

An alternative non-viral mechanism for delivery of factors to target cells includes using electric current to open pores as per devices and methods devised by MaxCyte, Gaithersburg, Md.

In other embodiments, target cells are genetically engineered to over-express a target transcription factor(s) (or other like signal transduction modifying factor), for example, by transiently or stably transfecting the target transcription factor DNA into a stem cell. Aspects of the invention include using suitable vectors to transport and express target transcription factor DNA into target stem cells.

Note that delivery mechanisms can include combinations of non-viral and viral techniques. For example, genetically engineering a target cell to overexpress a useful factor, the once modified cells then further modified by treatment with factor loaded polymeric vesicles. In general target cells of the invention can be modified one or more times through genetic engineering techniques and one or more additional times using various non-viral techniques.

These modified target cells (either non-virally or virally modified) are then used in subsequent therapeutic applications, including: the in vivo or in vitro growth and replacement of cartilage, bone, pancreas, intervetebral disc, heart muscle, and the like. In each of the above embodiments target cell lines having enhanced therapeutic applicability, for example, stem cells that have been modified to include greater numbers of phosphorylated SMAD proteins (1, 5 and/or 8), will differentiate into a target connective tissue. These embodiments can be performed in the presence or absence of treatment with bone morphogenetic protein or other like factors. In general, therefore, modified target cells have enhanced capacity for a therapeutic application.

### Smads

There are at least 5 mammalian receptor-mediated Smad proteins—Smad1, Smad2, Smad3, Smad5, and Smad8, each of which acts as a substrate for the TGFβ family of receptors. Smads 1, 5 and 8 are activated by bone morphogenetic protein (BMP) to accumulate in the nucleus and modulate transcription of genes necessary to cause differentiation of MSCs into chondrocytes and osteoblasts. Smads 1, 5 and 8 have been shown to accumulate in the nucleus as a result of receptor-mediated phosphorylation events with or without subsequent binding to Smad4. Randall, R, et al. Cellular Biology, 2004, 1106-1121; Shi and Massagué, Cell 2003; 113:685-700. These phosphorylation events are triggered by activation of cell surface TGFβ receptors (by BMP) thereby forming hetero-tetrameric receptor complexes capable of serine/threonine kinase activity. In general, phosphorylation at the C terminus of Smads 1, 5, and 8 creates a pS-x-pS motif recognized by DNA-binding cofactors that then result in gene selectivity and influence the recruitment of either transcriptional coactivators or corepressors. Shi et al., Cell 1998; 94:585-594.

Smad4 is predominately a cytoplasmicly located protein which interacts with phosphorylated Smad1, 2, 3, 5 and/or 8 to form complexes that migrate into the nucleus. These Smad-based complexes interact with target transcription factors in the nucleus to modulate target gene expression.

In general, Smad polypeptides of the invention can be prepared using various recombinant expression techniques or can be isolated as native protein from various target tissue. It is envisioned that either recombinant or native Smad proteins can be used in the cell modification methods of the invention. Where the Smad proteins are recombinant, proteins can be formed as fusion proteins for facilitating purification of the recombinately expressed Smads—for example, the heterologous protein can be a histidine tag or GST tag.

With regard to native Smad proteins, target Smad polypeptides can be isolated from a starting tissue source. Preferably, the starting tissue source is pre-treated with BMP-2 or other like signaling molecule that results in phosphorylation of the Smad proteins. The phosphorylated Smad protein is then isolated using techniques known in the art in a phosphorylated form, for example using anti-SMAD antibodies in immunoisolation procedures. Funaba M., et al. Recombinant Expression and Purification of Smad Proteins. Protein Expression and Purification, Vol. 20, No. 3, December 2000, pp. 507-513(7).

In particular, embodiments of the invention provide recombinant, phosphorylated Smad1, Smad5 and Smad8 polypeptides for inclusion into target cells, for example MSC or osteoblasts. Phosphorylated Smad proteins can be incorporated into target cells via any one of the variety of protein inclusion technologies described above. For example, phosphorylated Smad proteins can be delivered into cells using polymeric vesicles, liposomes, other like lipid vesicle based procedure, protein transduction domains (PTDs) or via electric current technology.

Note that embodiments of the invention also include the use of non-phosphorylated, receptor activated Smad proteins and non-receptor activated Smad proteins such as Smad4. In these embodiments the target cells are modified with the unphosphorylated molecules. The target cells can then be treated in a matter such that the increased levels of unphosphorylated, receptor activated Smad proteins become phosphorylated before use in the therapeutic application.

As briefly mentioned above, Smad proteins of the invention can be co-aggregated into transcription mediating complexes prior to incorporation into target cells. For example, Smad proteins can be pre-incubated together to form transcription activating complexes that are then loaded into polymeric vesicles and incorporated into a target cell. These complexes can undergo in vitro phosphorylation to ensure proper function once incorporated into the target cell or can be isolated as native complexes from appropriately treated cells.

### Other Transcription Factors

The above described methods and compositions can also be used to increase the relative number of transcription factors in the nucleus of a target cell. For example, core binding factor alpha 1 (Cbfa1) is a known transcription factor involved with osteoblast differentiation. A relative increase in Cbfa1 amounts in the nucleus of a target cell facilitate the differentiation of the modified cell into osteoblasts in both an in vitro and in vivo context. As discussed above, using methods and compositions discussed above Cbfa1 can be expressed as a recombinant protein or isolated as a native protein. Cbfa1 expression is as described in Kojima H., et al., Strong and Rapid Induction of Osteoblast Differentiation by Cbfa1/Til-1 Overexpression for Bone Regeneration. J. Biol. Chem., Vol. 280, Issue 4, 2944-2953, Jan. 28, 2005.

In general, the following references are cited and incorporated by reference herein (cited references provide additional description for useful transcription factors of the present invention):

B K Hall, 1988, AM. Sci. 76:174-181;

Kojima H. et al., J. Biol. Chem., Vol. 280, Issue 4, 2944-2953, Jan. 28, 2005;

Alliston T. et al., The EMBO Journal (2001) 20, 2254-2272;

Leboy P., Annals of the New York Academy of Sciences, Vol. 1068, p. 14, April 2006; and

Kurisaki K. et al., Mol. Cell. Boil. 2003, Vol. 23, n13, pp. 4494-4510.

In some embodiments, multiple modifications can be made to the target cell to prepare a cell that has increased capacity to differentiate into the differentiated cell necessary in the therapeutic application.

### Vectors

Embodiments of the invention provide vectors containing polynucleotide molecules useful in expressing agents useful in modifying signal transduction pathways within target cells. Any of the polynucleotide molecules of the invention may be contained in a vector, which generally includes a selectable marker and an origin of replication, for propagation in target cells. The vectors further include suitable transcriptional or translational regulatory sequences, such as those derived from a mammalian gene, operably linked to a SMAD polynucleotide molecule. Examples of such regulatory sequences include transcriptional promoters, operators, or enhancers, mRNA ribosomal binding sites, and appropriate sequences which control transcription and translation. Nucleotide sequences are operably linked when the regulatory sequence functionally relates to the DNA encoding the target protein. Thus, a promoter nucleotide sequence is operably linked to a SMAD encoding DNA sequence if the promoter nucleotide sequence directs the transcription of the SMAD sequences.

Selection of suitable vectors for cloning of SMAD or other like polynucleotide molecules encoding the target SMAD polypeptides of the invention will depend upon the target cell in which the vector will be transfected and expressed. Illustrative vector expression vehicles for mammalian cells are as follows: pCMV™-3 (Sigma-Alorich); CMV™-1 (Sigma-Alorich); pRK-5 Mammalian Expression Vector (BD Biosciences, Pharmigin), pLenti-DEST™ Gateway® vectors (Invitrogen), and the like.

The polypeptides of the invention may also be fusion proteins that include regions from heterologous proteins. Such regions may be included to allow, for example, improved stability, targeting to a organelle within the cell, etc. Illustrative vectors for use in this capacity include: HaloTag™ vectors (Promega) and CMV-GST (WARF).

The choice of a suitable expression vector for expression of signal transduction modifying agents will depend upon the specific target cell to be used. Examples of suitable expression vectors include pcDNA3.1/Hygro (Invitrogen) and pSVL (Pharmacia Biotech).

### Bulk Modified Stem Cell Production

In another aspect of the invention, Smad4 is isolated from target sources using various immunoprecipitation or other like techniques. The isolated Smad4 is loaded into a bank of allogenic stem cells by using non-viral delivery methods (including but not limited to polymeric vesicles, liposomes, electric current, protein transduction domains, etc). Stem cell lines can be obtained from Aldagen, ViaCell, Neoronyx, Geron, Advanced Cell Technology, BioE, etc. These modified cell lines are then typically frozen and saved for use in a therapeutic application as modified Smad4 cells. Note that the same general procedure can be performed with regard to any of the useful factors discussed herein thereby providing banks of modified stem cells for use in predetermined therapeutic applications.

### Therapeutic Applications

Modified target cells of the invention can be formulated as pharmaceutical compositions and administered to a host (patient in need thereof), preferably mammalian host, including a human patient, in a variety of forms adapted to the chosen route of administration. The compounds are preferably administered in combination with a pharmaceutically acceptable carrier, and may be combined with or conjugated to specific delivery agents. In addition, modified target cells can be administered to a patient in conjunction with a matrix necessary to facilitate the growth and/or differentiation of the target cell. For example, Smad modified MSCs for use in chondrocyte replacement can be administered with collagen, fibrin, a combination of collagen and fibrin or other like combinations.

Administration of the modified cells to a patient can be directly to the site of need, e.g., implanted into a surgical site, or can be via injection or infusion. For administration as injectable solutions or suspensions, the compositions can be formulated according to techniques well-know in the art, using suitable dispersing or wetting and suspending agents, such as sterile oils, including synthetic mono or diglycerides, and fatty acids, including oleic acid.

In one embodiment of the invention, the compounds may be administered directly to a target site, or by systemic delivery by intravenous injection.

Solutions or suspensions of the compounds can be prepared in water, isotonic saline (PBS) and optionally mixed with a nontoxic surfactant. Dispersions may also be prepared in glycerol, liquid polyethylene, glycols, DNA, vegetable oils, triacetin and mixtures thereof. Under ordinary conditions of storage and use, these preparations may contain a preservative to prevent the growth of microorganisms.

Note also that modified cells of the invention can be administered to a patient in combination with useful signaling molecules, for example BMP-2, cytokines, and the like.

Within the application, unless otherwise stated, the techniques utilized may be found in any of several well-known references, such as: Molecular Cloning: A Laboratory Manual (Sambrook et al. (1989) Molecular cloning: A Laboratory Manual), Gene Expression Technology (Methods in Enzymology, Vol. 185, edited by D. Goeddel, 1991 Academic Press, San Diego, Calif.), “Guide to Protein Purification” in Methods in Enzymology (M. P. Deutscher, 3d., (1990) Academic Press, Inc.), PCR Protocols: A Guide to Methods and Applications (Innis et al: (1990) Academic Press, San Diego, Calif.), Culture of Animal Cells: A Manual of Basic Technique, 2^{nd} ed. (R. I. Freshney (1987) Liss, Inc., New York, N.Y.), and Gene Transfer and Expression Protocols, pp 109-128, ed. E. J. Murray, The Humana Press Inc., Clifton, N.J.).

Having generally described the invention, the same will be more readily understood by reference to the following examples, which are provided by way of illustration and are not intended as limiting:

### EXAMPLES

### Example 1

Smad1 and Smad4 Modified Mesenchymal Stem Cell

MSCs are isolated from adipose or bone marrow tissue and treated with BMP-2 (thereby causing Smad1, 5 and 8 to phosphorylate). Treated cells are homogenized and phosphorylated Smad1/Smad5 and Smad8 proteins isolated using an immunoprecipitation assay (Smad polyclonal antibody as per Santa Cruz Biotechnology). Smad4 proteins are isolated from the same cell source using antibodies as obtained using the methods of Hao et al., Am J Phsiol Heart Circ. Physiol. 279: H3020-H3030 (2000).

Isolated Smad (1, 5 and 8) and Smad4 are combined in polymeric vesicles to prepare Smad loaded polymeric vesicles. The Smad loaded polymeric vesicles are then cultured with MSCs to prepare Smad (1, 5 and 8), Smad4 modified MSCs. These cells are modified to have enhanced capacity to differentiate and function as chondrocytes or osteoblasts. Modified cells are then implanted with or without a suitable matrix such as collagen into a target joint site in need thereof. Note that the MSCs can be autologous, i.e., harvested from the patient that will receive the modified cells. Although not wanting to be bound by any one theory, the combination of phosphorylated Smad (1, 5 and 8) and Smad4 is believed to facilitate migration of the Smad proteins into the nucleus and activation of genes involved in target site differentiation.

### Example 2

Example 1: Smad1, Smad4 and Cbfa-1 Modified Mesenchymal Stem Cell

The following example is identical to example 1 except that polymeric vesicles are also loaded with recombinant or native Cbfa-1 transcription factor. As in the above example, the present example further provides Cbfa-1 to enhance Smad (1, 5 and 8) mediated transcription by also providing one of the relevant transcription factors known to operate in the Smad transcription initiation complex (for example on the osteocalcin promoter).

### Example 3

Electric Current Mediated Incorporation of Smad4 to Modify MSCs

MSCs are isolated from adipose or bone marrow tissue. MSCs are homogenized and Smad4 proteins are isolated from the cell source using antibodies as obtained using the methods of Hao et al., Am J Phsiol Heart Circ. Physiol. 279: H3020-H3030 (2000).

Isolated Smad4 is added to MSCs for implantation into a patient in need thereof. The MSCs are first treated with an electric current using the protocols as described by MaxCyte. These non-virally modified cells can be treated with BMP-2. The modified cells now have facilitated Smad4 levels for shuttling the BMP-2 mediated Smad1 into the nucleus for appropriate MSC differentiation.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

1. A method for preparing a modified cell for use in implantation into a patient in need thereof comprising:
loading a polymeric vesicle with at least one transcription factor; and
incubating the at least one transcription factor loaded polymeric vesicle with a cell to prepare a modified cell having an increased level of the at least one transcription factor as compared to the unmodified cell;
wherein the modified cell is implanted into the patient in need thereof.

2. The method of claim 1 wherein the at least one transcription factor is Smad4.

3. The method of claim 1 wherein the at least one transcription factor is Smad1.

4. The method of claim 3 wherein the Smad1 is phosphorylated.

5. The method of claim 1 wherein the modified cell is a modified mesenchymal stem cell.

6. The method of claim 5 wherein the modified mesenchymal stem cell is autologous for the patient.

7. The method of claim 1 further comprising treating the modified cell with BMP-2.

8. The method of claim 1 wherein the at least one transcription factor is Smad4 and Smad1.

9. The method of claim 1 wherein the at least one transcription factor is Smad4 and SMAD polypeptide co-factor.

10. The method of claim 9 wherein the SMAD polypeptide co-factor is P300.

11. A method for preparing a modified mesenchymal stem cell for use in implantation into a patient in need thereof comprising:
stably transfecting a target cell with a first transcription factor to prepare a modified mesenchymal stem cell having increased expression of the first transcription factor as compared to an unmodified mesenchymal stem cell;
loading a polymeric vesicle with at least one additional transcription factor; and
incubating the at least one additional transcription factor loaded polymeric vesicle with the modified mesenchymal stem cell to prepare a modified mesenchymal stem cell having both increased expression of the first transcription factor and an increased level of the at least one additional transcription factor as compared to the unmodified cell;
wherein the modified mesenchymal stem cell is implanted into the patient in need thereof.

12. The method of claim 11, wherein the first transcription factor and the additional transcription factor are independently selected from the group consisting of Smad 1, Smad4, Smad5, Smad8, and Cbfa-1.

13. The method of claim 11 further comprising treating the modified cell with BMP-2.

14. The method of claim 11 wherein the modified mesenchymal stem cell is autologous for the patient.

15. A method of inducing differentiation of a stem cell into a target tissue, the method comprising:
loading a polymeric vesicle with at least one transcription factor; and
incubating the at least one transcription factor loaded polymeric vesicle with the stem cell to prepare a modified stem cell having an increased level of the at least one transcription factor as compared to an unmodified stem cell;
wherein the modified stem cell differentiates into the target tissue.

16. The method of claim 15, wherein the stem cell is a mesenchymal stem cell.

17. The method of claim 15, wherein the transcription factor is selected from the group consisting of Smad 1, Smad4, Smad5, Smad8, and Cbfa-1.

18. The method of claim 15, wherein the stem cell is further modified to incorporate at least one Smad co-factor selected from the group consisting of P300, PCAF, GCN5, and MSG1.

19. The method of claim 15, wherein the stem cell is further modified to incorporate a protein involved in signal transduction selected from the group consisting of kinases, phosphatases, nuclear transport proteins, DNA binding proteins, and heat shock proteins.

20. A method of inducing differentiation of a cell into a target tissue for use in implantation into a patient in need thereof, the method comprising:
transporting at least one transcription factor inside the cell using a transport mechanism selected from the group consisting of polymeric vesicles, liposomes, protein transduction domains, and electricity induced pore opening, to prepare a modified cell having an increased level of the at least one transcription factor as compared to an unmodified cell;
wherein the modified cell differentiates into the target tissue; and
wherein the modified cell is implanted into the patient in need thereof.

21. The method of claim 20, wherein the modified cell has enhanced capacity to differentiate and/or function as a chondrocyte or osteoblast.
